(19) United States
(12) Patent Application Publication
(10) Pub. No.: US 2023/0038138 A1
(43) Pub. Date: Feb. 9, 2023

(54) COMBINATION THERAPY FOR TREATING CANCER

(71) Applicant: ASTRAZENECA AB, Sodertalje (SE)

(72) Inventors: Maureen HATTERSLEY, Wilmington, DE (US); Gayle Pageau POULIOT, Wilmington, DE (US); Huawei Raymond CHEN, Wilmington, DE (US); Serena DE VITA, Wilmington, DE (US)

(21) Appl. No.: 17/758,515

(22) PCT Filed: Jan. 8, 2021
(86) PCT No.: PCT/IB2021/050122

§ 371 Date: Jul. 8, 2022

Related U.S. Application Data
(60) Provisional application No. 62/958,792, filed on Jan. 9, 2020.

(51) Int. Cl. A61K 31/5025

(52) U.S. Cl. CPC A61K 31/5025

(57) ABSTRACT

The present disclosure relates, in general, to therapeutic combinations of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one and olaparib, and to corresponding methods of treatment, pharmaceutical compositions, and kits.

### RELATED APPLICATION

This application claims benefit of priority under 35 U.S.C. § 119(e) of the U.S. Provisional Application No. 62/958,792, filed Jan. 9, 2020, which is incorporated by reference herein in its entirety for all purposes.

### BACKGROUND

While much progress has been made in the treatment of cancer, many of these patients who have such cancers live with an incurable disease. There are many proteins and pathways involved in cancer and the research thereof has been advanced rapidly. Accordingly, it's important to continue to find new treatments for patients with incurable cancer.

### SUMMARY

In some embodiments, disclosed is a method of treating cancer in a human subject in need thereof, comprising administering to the human subject a first amount of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and a second amount of olaparib or a pharmaceutically acceptable salt thereof. In the method, the first amount and the second amount together comprise a therapeutically effective amount. Furthermore, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is administered under an intermittent dosing schedule and olaparib or a pharmaceutically acceptable salt thereof is administered under a continuous dosing schedule.

In some embodiments, disclosed is AZD5153 or a pharmaceutically acceptable salt thereof for use in the treatment of a cancer, wherein the treatment comprises intermittent administration of AZD5153 or a pharmaceutically acceptable salt thereof and continuous administration of olaparib or a pharmaceutically acceptable salt thereof.

In some embodiments, disclosed is olaparib or a pharmaceutically acceptable salt thereof for use in the treatment of a cancer, wherein the treatment comprises continuous administration of olaparib or a pharmaceutically acceptable salt and intermittent administration of AZD5153 or a pharmaceutically acceptable salt thereof.

In some embodiments, the cancer is selected from ovarian cancer, breast cancer, pancreatic cancer, and prostate cancer.

In some embodiments, the cancer is a relapsed or refractory cancer.

In some embodiments, the cancer is platinum-resistant or platinum-refractory.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered under an intermittent dosing schedule for a cycle of 21 days; and olaparib or a pharmaceutically acceptable salt thereof is orally administered under a continuous dosing schedule for a cycle of 21 days.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered on an intermittent dosing schedule for 7 to 14 consecutive days out of every 21 days. For example, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered for 7, 8, 9, 10, 11, 12, 13, or 14 consecutive days out of every 21 days and the remaining days of the 21-day cycle are void days.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered from day 1 through day 7 in a cycle of 21 days wherein day 8 through day 21 are void days.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered from day 1 through day 14 in a cycle of 21 days wherein day 15 through day 21 are void days.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered under an intermittent dosing schedule for a cycle of 7 days; and olaparib or a pharmaceutically acceptable salt thereof is orally administered under a continuous dosing schedule for a cycle of 7 days.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered on an intermittent dosing schedule for 3 to 5 consecutive days out of every 7 days. For example, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered for 3, 4 or 5 consecutive days out of every 7 days and the remaining days of the 7-day cycle are void days

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered from day 1 through day 3 in a cycle of 7 days wherein day 4 through day 7 are void days.

In some embodiments, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered from day 1 through day 5 in a cycle of 7 days wherein day 6 and day 7 are void days.

In some embodiments, about 5 mg to about 15 mg of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered to the human subject once or twice daily. In some embodiments, about 5 mg or about 10 mg of 3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered to the human subject once or twice daily. For example, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof can be orally administered about 5 mg once daily, about 5 mg twice daily, about 10 mg once daily, about 10 mg twice daily, about 15 mg once daily, or about 15 mg twice daily.

In some embodiments, about 200 mg to about 300 mg olaparib or a pharmaceutically acceptable salt thereof is orally administered to the human subject twice daily. In some embodiments, about 200 mg or about 300 mg olaparib or a pharmaceutically acceptable salt thereof is orally administered to the human subject twice daily. For example, olaparib or a pharmaceutically acceptable salt thereof is orally administered about 200 mg twice daily or about 300 mg twice daily.

In some embodiments, disclosed is a kit comprising a first pharmaceutical composition comprising (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and a pharmaceutically acceptable carrier; and a second pharmaceutical composition comprising olaparib or a pharmaceutically acceptable salt thereof and instructions for use.

### BRIEF DESCRIPTIONS OF THE DRAWING

FIG. 1 illustrates the reduction in tumor volume over time following treatment with AZD5153 alone, olaparib alone, and the combinations of AZD5153 and olaparib on intermittent and continuous dosing schedules in an OVCAR3 xenograft model.

### DETAILED DESCRIPTION

In some embodiments, disclosed is a method of treating cancer by a combination therapy of AZD5153 and olaparib. In some embodiments, the method comprises administering to a subject in need thereof a first amount of AZD5153 or a pharmaceutically acceptable salt thereof and a second amount of olaparib or a pharmaceutically acceptable salt thereof, wherein the first amount and the second amount together comprises a therapeutically effective amount.

The term “AZD5153” refers to a compound with the chemical name of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one and structure shown below:

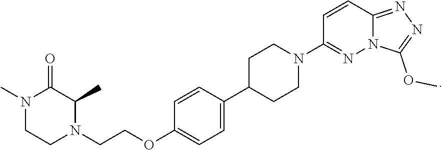

AZD5153 is a bivalent triazolopyridazine based bromodomain and extraterminal (BET) inhibitor. Bromodomain-containing proteins are implicated in diverse diseases and are of substantial interest as therapeutic targets. The BET family consists of four proteins known as BRD2, BRD3, BRD4, and BRDT, each of which contains two separate bromodomains. BRD4 in particular has been considered as a potential drug target for treating cancer (e.g., hematological malignancies).

Preparation and use of AZD5153 is disclosed in International Application Publication No. WO 2016/016618, the content of which is hereby incorporated by reference in its entirety. In some embodiments, a free base AZD5153, i.e., (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one, is administered to a subject. In some embodiments, a pharmaceutically acceptable salt of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one is administered to a subject. In some embodiment, a crystalline AZD5153 is administered to a subject. In some embodiments, a co-crystal of AZD5153 is administered to a subject, wherein the co-former molecule is 6-hydroxy-2-naphthoic acid. Examples of crystalline AZD5153 and co-crystal of AZD5153 are described in WO 2016/016618.

Olaparib is described chemically as 4-[(3-{[4-(cyclopropylcarbonyl)piperazin-1-yl]carbonyl}-4- fluorophenyl)methyl]phthalazin-1(2H)-one and has the following chemical structure:

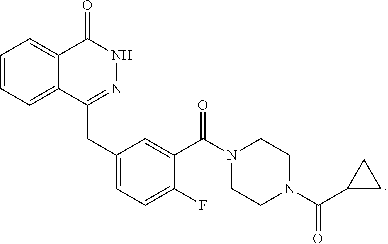 [SMILES: O=C(c1cc(Cc2n[nH]c(=O)c3ccccc23)ccc1F)N1CCN(C(=O)C2CC2)CC1]

Olaparib is an orally bioavailable inhibitor of poly (ADP-ribose) polymerase (PARP) enzymes, including PARP1, PARP2, and PARP3. PARP enzymes are involved in normal cellular functions, such as DNA transcription and DNA repair. Olaparib has been shown to inhibit growth of select tumor cell lines in vitro and decrease tumor growth in mouse xenograft models of human cancer, both as monotherapy or following platinum based chemotherapy. Increased cytotoxicity and anti-tumor activity following treatment with olaparib were noted in cell lines and mouse tumor models with deficiencies in BRCA (breast cancer gene) and non-BRCA proteins involved in the homologous recombination repair (HRR) of DNA damage and correlated with platinum response. In vitro studies have shown that olaparib-induced cytotoxicity may involve inhibition of PARP enzymatic activity and increased formation of PARP-DNA complexes, resulting in DNA damage and cancer cell death.

Preparation and use of olaparib is disclosed in International Application Publication No. WO/2004/080976, the content of which is hereby incorporated by reference in its entirety. In some embodiments, a free base olaparib is administered to a subject. In some embodiments, a pharmaceutically acceptable salt of olaparib is administered to a subject. In some embodiments, a crystalline olaparib or a pharmaceutically acceptable salt of olaparib is administered to a subject. In some embodiments, an oral tablet comprising a solid dispersion of olaparib in a matrix co-polymer is administered to a subject. In certain embodiments the oral tablet comprises a solid dispersion of olaparib in the matrix co-polymer copovidone. Preparation of such solid dispersions and oral tablets is disclosed in International Application Publication No. WO2010/041051, the content of which is hereby incorporated by reference in its entirety.

In some embodiments, AZD5153 or a pharmaceutically acceptable salt thereof and olaparib or a pharmaceutically acceptable salt thereof are administered separately, sequentially or simultaneously in a treatment cycle. In some embodiments, AZD5153 or a pharmaceutically acceptable salt thereof is intermittently administered in the treatment cycle and olaparib or a pharmaceutically acceptable salt thereof is continuously administered in the treatment cycle. The term “intermittent” or “intermittently” as used herein means stopping and starting administration of a therapeutic agent, e.g. AZD5153, at either regular or irregular intervals in a treatment cycle. For intermittent administration, there is at least one void day in the treatment cycle. By “void day”, it is meant a day when AZD5153 is not administered. The term “continuous” or “continuously” refers to administration of a therapeutic agent, e.g. olaparib, at regular intervals without stopping or interruption, i.e., no void day.

A “cycle”, “treatment cycle” or “dosing schedule”, as used herein, refers to a period of combination treatment that is repeated on a regular schedule. For example, the treatment can be given for one week, two weeks, or three weeks wherein AZD5153 and olaparib are administered in a coordinated fashion. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months. In some embodiments, the period of rest, i.e., void day(s), in a treatment cycle is about 1 day to about 1 month. In some embodiments, the period of rest in a treatment cycle is about 1 day, about 3 days, about 5 days, about 1 week, about 2 weeks, or about 3 weeks.

In some embodiments, AZD5153 or a pharmaceutically acceptable salt thereof and olaparib or a pharmaceutically acceptable salt thereof are administered to the human subject in one or more treatment cycles, e.g., a treatment course. A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

In some embodiments, AZD5153 or a pharmaceutically acceptable salt thereof is administered for 7 to 14 days (e.g., 7, 8, 9, 10, 11, 12, 13 or 14 days) in a 21-day treatment cycle, while olaparib or a pharmaceutically acceptable salt thereof is administered for 21 days in the 21-day treatment cycle. In some embodiments, AZD5153 or a pharmaceutically acceptable salt thereof is administered for 7 or 14 days in a 21-day treatment cycle, while olaparib or a pharmaceutically acceptable salt thereof is administered for 21 days in the 21-day treatment cycle. In some embodiments, AZD5153 or a pharmaceutically acceptable salt thereof is administered for 3 to 5 days (e.g., 3, 4 or 5 days) in a 7-day treatment cycle, while olaparib or a pharmaceutically acceptable salt thereof is administered for 7 days in the 7-day treatment cycle.

In some embodiments, AZD5153 or a pharmaceutically acceptable salt thereof is administered on day 1 through day 7 in a 21-day treatment cycle, while olaparib or a pharmaceutically acceptable salt thereof is administered on day 1 through day 21 in the 21-day treatment cycle. In some embodiments, AZD5153 or a pharmaceutically acceptable salt thereof is administered on day 1 through day 14 in a 21-day treatment cycle, while olaparib or a pharmaceutically acceptable salt thereof is administered on day 1 through day 21 in the 21-day treatment cycle. In some embodiments, AZD5153 or a pharmaceutically acceptable salt thereof is administered on day 1 through day 5 in a 7-day treatment cycle, while olaparib or a pharmaceutically acceptable salt thereof is administered on day 1 through day 7 in the 7-day treatment cycle. In some embodiments, AZD5153 or a pharmaceutically acceptable salt thereof is administered on day 1 through day 3 in a 7-day treatment cycle, while olaparib or a pharmaceutically acceptable salt thereof is administered on day 1 through day 7 in the 7-day treatment cycle.

In some embodiments, AZD5153 is administered orally. In some embodiments, AZD5153 is administered in a dose of up to about 40 mg (for example, up to about 5 mg, up to about 10 mg, up to about 15 mg, up to about 20 mg, up to about 25 mg, up to about 30 mg, up to about 35 mg, or up to about 40 mg AZD5153) per day. AZD5153 can be administered either once a day (QD) or twice a day (BID). In some embodiments, AZD5153 is administered in a dose of about 5 mg BID, about 10 mg QD, about 10 mg BID, or about 15 mg QD.

In some embodiments, olaparib or a pharmaceutically acceptable salt thereof is administered orally. In some embodiments, olaparib or a pharmaceutically acceptable salt thereof is in capsule dosage form. In some embodiments, olaparib or a pharmaceutically acceptable salt thereof is in tablet dosage form. In some embodiments, olaparib or a pharmaceutically acceptable salt thereof is administered in a dose of about 300 mg orally twice per day. In some embodiments, olaparib or a pharmaceutically acceptable salt thereof is administered in a dose of about 200 mg orally twice daily.

In some embodiments, AZD5153 and olaparib are administered orally to a subject for treating cancer in a 21-day treatment cycle, wherein AZD5153 is administered in a dose of about 5 mg BID, 10 QD, 10 mg BID, 15 mg QD, or 15 mg BID on day 1 through day 7 in the treatment cycle, and olaparib is administered in a dose of about 200 mg or 300 mg BID on day 1 through day 21 in the treatment cycle.

In some embodiments, AZD5153 and olaparib are administered orally to a subject for treating cancer in a 21-day treatment cycle, wherein AZD5153 is administered in a dose of about 5 mg BID, 10 QD, 10 mg BID, 15 mg QD, or 15 mg BID on day 1 through day 14 in the treatment cycle, and olaparib is administered in a dose of about 200 mg or 300 mg BID on day 1 through day 21 in the treatment cycle.

In some embodiments, AZD5153 and olaparib are administered orally to a subject for treating cancer in a 7-day treatment cycle, wherein AZD5153 is administered in a dose of about 5 mg BID, 10 QD, 10 mg BID, 15 mg QD, or 15 mg BID on day 1 through day 5 in the treatment cycle, and olaparib is administered in a dose of about 200 mg or 300 mg BID on day 1 through day 7 in the treatment cycle.

In some embodiments, AZD5153 and olaparib are administered orally to a subject for treating cancer in a 7-day treatment cycle, wherein AZD5153 is administered in a dose of about 5 mg BID, 10 QD, 10 mg BID, 15 mg QD, or 15 mg BID on day 1 through day 3 in the treatment cycle, and olaparib is administered in a dose of about 200 mg or 300 mg BID on day 1 through day 7 in the treatment cycle.

The language “treat,” “treating” and “treatment” includes the reduction or inhibition of enzyme or protein activity related to BRD4, PARP or cancer in a subject, amelioration of one or more symptoms of cancer in a subject, or the slowing or delaying of progression of cancer in a subject. The language “treat,” “treating” and “treatment” also includes the reduction or inhibition of the growth of a tumor or proliferation of cancerous cells in a subject.

The language “inhibit,” “inhibition” or “inhibiting” includes a decrease in the baseline activity of a biological activity or process.

The term “cancer” includes, but is not limited to a disease caused by an uncontrolled division of abnormal cells in a part of the body. In some embodiments, the cancer includes cancers that are susceptible to treatment with BRD4 inhibitors (e.g., AZD5153). In some embodiments, the cancer includes cancers that are susceptible to treatment with PARP inhibitors (e.g., olaparib). In some embodiments, the cancer is ovarian cancer, breast cancer, pancreatic cancer, and prostate cancer. In some embodiments, the cancer is relapsed or refractory cancer. In some embodiments, the cancer is platinum-resistant or platinum-refractory cancer.

The language “pharmaceutical composition” includes compositions comprising an active ingredient and a pharmaceutically acceptable excipient, carrier or diluent, wherein the active ingredient is AZD5153 or a pharmaceutically acceptable salt thereof, or olaparib or a pharmaceutically acceptable salt thereof. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. In some embodiments, the pharmaceutical compositions are in solid dosage forms, such as capsules, tablets, granules, powders, sachets, etc. In some embodiments, the pharmaceutical compositions are in the form of a sterile injectable solution in one or more aqueous or non-aqueous non-toxic parenterally-acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The term “subject” includes warm-blooded mammals, for example, primates, dogs, cats, rabbits, rats, and mice. In some embodiments, the subject is a primate, for example, a human. In some embodiments, the subject is suffering from cancer, such as ovarian cancer, breast cancer, pancreatic cancer, and prostate cancer. In some embodiments, the subject is suffering from ovarian cancer or breast cancer. In some embodiments, the subject is suffering from relapsed or refractory ovarian cancer. In some embodiments, the subject is suffering from relapsed or refractory breast cancer. In some embodiments, the subject is suffering from cancer and is treatment naïve (e.g., has never received treatment for cancer). In some embodiments, the subject is suffering from cancer and is platinum-resistant or platinum-refractory. Platinum-resistant disease is defined by progression within 6 months following the last administered platinum-based regimen. Platinum-refractory disease is defined by lack of at least a partial response while on platinum-containing regimens. A platinum-based regimen includes drugs that contain the metal platinum, such as cisplatin and carboplatin.

The language “therapeutically effective amount” includes that amount of AZD5153 and that amount of olaparib which together will elicit a biological or medical response in a subject, for example, the reduction or inhibition of enzyme or protein activity related to PARP, BET including one or more of BRD2, BRD3, BRD4, and BRDT, or cancer; amelioration of symptoms of cancer; or the slowing or delaying of progression of cancer. In some embodiments, the language “therapeutically effective amount” includes the amount of AZD5153 and olaparib together that is effective to at least partially alleviate, inhibit, and/or ameliorate cancer or inhibit BRD4 or PARP, and/or reduce or inhibit the growth of a tumor or proliferation of cancerous cells in a subject.

In some embodiments, disclosed is a kit comprising: a first pharmaceutical composition comprising AZD5153 or a pharmaceutically acceptable salt thereof and a pharmaceutically acceptable carrier; and a second pharmaceutical composition comprising olaparib or a pharmaceutically acceptable salt; and instructions for using the first and second pharmaceutical compositions in combination. In some embodiments, the first pharmaceutical composition comprises a first amount of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and the second pharmaceutical composition comprises a second amount of olaparib or a pharmaceutically acceptable salt thereof; wherein the first amount and the second amount together comprise a therapeutically effective amount.

In some embodiments, disclosed is a pharmaceutical product comprising i) (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and ii) olaparib or a pharmaceutically acceptable salt thereof. In some embodiments of the pharmaceutical product, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and olaparib or a pharmaceutically acceptable salt thereof are present in a single dosage form. In some embodiments of the pharmaceutical product, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and olaparib or a pharmaceutically acceptable salt thereof are present in separate dosage forms.

### EXAMPLES

### Example 1. Efficacy of AZD5153, a Bivalent BRD4 Inhibitor, Combined with Olaparib in a Pre-Clinical Model of Ovarian Cancer

OVCAR3: NIH-OVCAR3 human ovarian tumor cells (15×10⁶/mouse) were implanted subcutaneously in female CB.17 SCID mice. The mice were randomized into groups of eight for efficacy, based on tumor volume and treated with either vehicle (0.5% HPMC/0.1% Tween 80), AZD5153; olaparib or the combination of AZD5153 and olaparib approximately 10 days after implantation. AZD5153 was formulated in 0.5% HPMC/0.1% Tween 80 and dosed orally for 42 days. Olaparib was formulated in 50% of 30% w/v HP-β-CD (Kleptose) in purified water and also dosed for 42 days. For the combination groups, olaparib was dosed daily for 42 days and AZD5153 was dosed either daily, for 7 days on and 14 days off for 2 cycles or 14 days on and 7 days off for 2 cycles. Tumor length and width was measured by caliper and tumor volume was calculated using the formula (length×width²)*π/6 then reported as tumor volume.

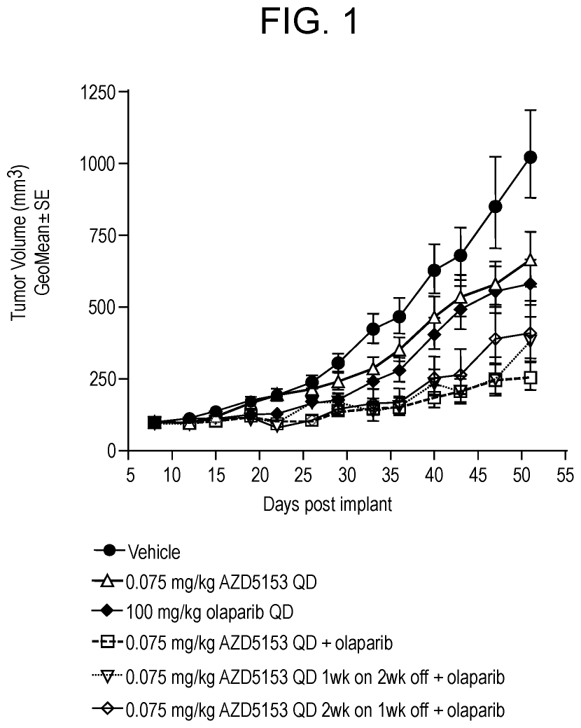

Results: As shown in FIGS. 1, both AZD5153 and olaparib monotherapy were modestly efficacious in the OVCAR3 ovarian model, the combination of agents demonstrated markedly stronger efficacy and combination efficacy is maintained with intermittent dosing of AZD5153 and is comparable to daily dosing with AZD5153.

### Example 2. Clinical Study of Combination to Treat Relapsed/Refractory Ovarian, Pancreatic, Triple Negative Breast or Prostate-Cancer

### Inclusion Criteria Specific for MTD Monotherapy Expansion and Olaparib Combination Dose Escalation (HGSO Cancer Patients)

Histologically or cytologically confirmed platinum-resistant or platinum-refractory HGSO, fallopian, or primary peritoneal cancer in the relapsed setting. Platinum-resistant disease is defined by progression within 6 months following the last administered platinum-based regimen. Platinum-refractory disease is defined by lack of at least a partial response while on platinum-containing regimens.
Triple-negative breast cancer (TNBC) that is refractory to standard therapy(ies) or for which no standard therapy(ies) exists
Refractory metastatic castrate-resistant prostate cancer (mCPRC)
Pancreatic ductal adenocarcinoma (PDAC) with progression on standard treatment
Patients with histologically or cytologically confirmed platinum-resistant OR platinum-refractory HGSO (high grade serous ovarian) in the relapsed setting must have at least 1 lesion that can be accurately assessed at baseline by CT scan according to RECIST v1.1 criteria (Eisenhauer et al. 2009) and is suitable for repeated assessment. Platinum-resistant disease is defined by progression within 6 months following the last administered platinum-based regimen. Platinum-refractory disease is defined by lack of at least a partial response while on platinum-containing regimens.
Patients will be allowed on study regardless of their BRCA mutational status. Information about their BRCA mutational status must be collected if patients have been tested as part of their standard of care.
Previous response to olaparib or other PARP inhibitor is not required.
Patients must have at least 1 site of disease amenable to biopsy at screening, and must be a candidate for tumor biopsy. Patients must be willing to undergo a new tumor biopsy during screening. An on-treatment tumor biopsy will be mandatory in the olaparib combination dose escalation portion of this study, and optional from consenting patients in the monotherapy expansion at MTD (maximum tolerated dose).
For the olaparib combination portion of this study, patients are required to have hemoglobin ≥10 g/dL at study entry.

### AZD5153+Olaparib Combination Dose Escalation

The combination of AZD5153 with olaparib will start at the doses of 10 mg BID of AZD5153 and 300 mg BID of olaparib. Combination dosing will proceed over 21-day cycle periods until criteria for discontinuation are met.

Subsequent dose levels of AZD5153 in combination with 300 mg BID olaparib will be chosen based on the safety data emerging from both the ongoing monotherapy dose escalation and from the combination cohorts already dosed.

An example of the dose escalation for AZD5153 and olaparib with a fixed dose of olaparib 300 mg BID is shown in Table 2. Additional dose levels or alternative dosing schedules may be evaluated and recommended by the SRC (safety review committee) based on evolving safety and PK data from already concluded cohorts, including AZD5153 monotherapy cohorts. The dose level of AZD5153 in combination with olaparib will not exceed the declared monotherapy MTD and dose increments between subsequent cohorts will not exceed two-fold.

If the dose of 10 mg BID is not tolerated upon continuous dosing, intermittent schedules (i.e. 2 weeks on/1 week off, 3 days on/4 days off, or additional schedules) might be explored to mitigate toxicity based on SRC feedback.

TABLE 1
Example of a dose escalation scheme for AZD5153 and olaparib

| Dose | | |
|---|---|---|
| Cohort | AZD5153 dose | olaparib dose |
| Cohort 1 | 10 mg BID | 300 mg BID |
| Cohort 2 | 15 mg BID | 300 mg BID |
| Cohort 3 | 10 mg QD 2 weeks on, 1 week off | 300 mg BID |

Dose escalation and de-escalation will follow the Bayesian Adaptive Design schema below:
A minimum of 3 patients will be recruited to the first combination cohort and will be treated with a combination dose of AZD5153 (10 mg BID) and olaparib 300 mg BID.
If no DLTs (dose limiting toxicities) are seen, then subsequent cohorts will be opened with the AZD5153 dose escalated in line with that which has been shown as safe in the monotherapy but not exceeding doubling. The olaparib dose will remain at 300 mg BID.
If the escalation of the AZD5153 component reaches the monotherapy MTD without incurring DLTs, then the combination MTD will be declared to be the same as the monotherapy MTD in AZD5153 with 300 mg BID of olaparib.
If at least one DLT is observed in any cohort, in combination with 300 mg BID olaparib, then CRM modelling will be used in the same manner as for monotherapy (Section 5.1.4).
If needed, a Bayesian Logistic Regression Model, i.e., BLRM (Neuenschwander et al 2015), may be applied to better understand the toxicity across dose combinations. If this happens then the dose escalation may end when:
   All allowable combinations are predicted to be toxic;
   All allowable combinations are predicted to be safe;
   The number of patients treated reaches a maximum of approximately 18; and
   The SRC considers that the combination MTD has been identified with sufficient confidence.
The AZD5153 component will not be escalated above the estimated monotherapy MTD (or the highest dose tested in monotherapy) and the olaparib component will not be escalated above 300 mg BID.

Patients will be enrolled in each combination dose level. The number of patients in this portion of the study will depend on the number of patients in each cohort and the number of cohorts.

Preliminary data for 11 patients were analyzed, and 3 patients treated with AZD5153 (10 mg BID) and olaparib (300 mg BID) were reported to have experienced DLTs of thrombocytopenia following combination treatment in Cycle 1. The one patient experienced an AE of thrombocytopenia Grade 3 that subsequently resolved after dose interruption. Another patient experienced an AE of thrombocytopenia Grade 4 that also resolved after dose interruption; the patient restarted treatment at a reduced dose (AZD5153 5 mg BID+olaparib 300 mg BID). The third patient experienced an AE of thrombocytopenia Grade 4 that resolved after dose interruption, but recurred twice as Grade 2 and then Grade 3 that was last recorded as not recovered/not resolved. None of the patients discontinued from AZD5153 or olaparib in association with an AE.

This written description uses examples to disclose the invention and to enable any person skilled in the art to practice the invention, including making and using any of the disclosed salts, substances, or compositions, and performing any of the disclosed methods or processes. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims. While preferred embodiments of the invention are shown and described in this specification, such embodiments are provided by way of example only and are not intended to otherwise limit the scope of the invention. Various alternatives to the described embodiments of the invention may be employed in practicing the invention. Section headings as used in this section and the entire disclosure are not intended to be limiting.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicants reserve the right to challenge the accuracy and pertinence of the cited references.

1. A method of treating cancer in a human subject in need thereof, comprising administering to the human subject a first amount of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and a second amount of olaparib or a pharmaceutically acceptable salt thereof; wherein the first amount and the second amount together comprise a therapeutically effective amount; and (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is administered under an intermittent dosing schedule and olaparib or a pharmaceutically acceptable salt thereof is administered under a continuous dosing schedule.

2. The method of claim 1, wherein the cancer is selected from ovarian cancer, breast cancer, pancreatic cancer, and prostate cancer.

3. The method of claim 1, wherein the cancer is a relapsed or refractory cancer.

4. The method of claim 1, wherein the cancer is platinum-resistant or platinum-refractory.

5. The method of claim 1, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered under an intermittent dosing schedule for a cycle of 21 days; and olaparib or a pharmaceutically acceptable salt thereof is orally administered under a continuous dosing schedule for a cycle of 21 days.

6. The method of claim 5, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered on an intermittent dosing schedule for 7 to 14 consecutive days out of every 21 days.

7. The method of claim 5, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered on an intermittent dosing schedule for 7 or 14 consecutive days out of every 21 days.

8. The method of claim 1, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered under an intermittent dosing schedule for a cycle of 21 days; and olaparib or a pharmaceutically acceptable salt thereof is orally administered under a continuous dosing schedule for a cycle of 7 days.

9. The method of claim 8, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered on an intermittent dosing schedule for 3 to 5 consecutive days out of every 7 days.

10. The method of claim 9, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered on an intermittent dosing schedule for 3 or 5 consecutive days out of every 7 days.

11. The method of claim 1, wherein about 5 mg to about 15 mg of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof is orally administered to the human subject once or twice daily.

12. The method of claim 1, wherein about 200 mg to about 300 mg olaparib or a pharmaceutically acceptable salt thereof is orally administered to the human subject twice daily.

13.-17. (canceled)

18. A kit comprising:
a first pharmaceutical composition comprising (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and a pharmaceutically acceptable carrier; and
a second pharmaceutical composition comprising olaparib or a pharmaceutically acceptable salt thereof and instructions for use.

19. A pharmaceutical product comprising i) (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and ii) olaparib or a pharmaceutically acceptable salt thereof.

20. The pharmaceutical product of claim 19, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and olaparib or a pharmaceutically acceptable salt thereof are present in a single dosage form.

21. The pharmaceutical product of claim 19, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and olaparib or a pharmaceutically acceptable salt thereof are present in separate dosage forms.
